(19) United States
(12) Patent Application Publication
(10) Pub. No.: US 2015/0153343 A1
(43) Pub. Date: Jun. 4, 2015

(54) DETECTION SYSTEM FOR THE IDENTIFICATION OF INSECTICIDE RESISTANCE

(71) Applicant: BAYER CROPSCIENCE AG, Monheim (DE)

(72) Inventors: Ralf Nauen, Langenfeld (DE); Klaus Raming, Leverkusen (DE); Katharina Woelfel, Langenfeld (DE)

(21) Appl. No.: 14/405,807

(22) PCT Filed: Jun. 5, 2013
(86) PCT No.: PCT/EP2013/061619

§ 371 Date: Dec. 5, 2014

Related U.S. Application Data
(60) Provisional application No. 61/657,173, filed on Jun. 8, 2012.

(30) Foreign Application Priority Data
Jun. 8, 2012 (EP) 12004357.5
Sep. 12, 2012 (EP) 12184005.2

(51) Int. Cl. G01N 33/573

(52) U.S. Cl. CPC G01N 33/573; G01N 2333/90245

(57) ABSTRACT

A rapid, easy, and highly sensitive detection system is herein described for the identification of resistance of animal plant pests to neonicotinoids and pymetrozine. Further, a method for controlling insect pests, in particular resistant pests is provided.

### FIELD OF THE INVENTION

This invention relates to a detection kit for the easy identification of insecticide resistance in insects. The kit is in particular suitable for the detection of resistance in the field as it allows a quick and simple identification of a resistance marker with a sample size comprising a few insects. Further, the invention relates to a method for resistance management in insects, that are resistant to certain insecticides. The method and the kit are in particular suited for the cotton whitefly, Bemisia tabaci (Hemiptera: Aleyrodidae).

### BACKGROUND ART

It is known that resistance to the neonicotinoid insecticide imidacloprid in cotton whitefly (Bemisia tabaci) is associated with high expression levels of the cytochrome P450 monooxygenase CYP6CM1 (Insect Biochemistry and Molecular Biology 38, (2008), 634-644).

### Problem

A problem of growing importance in agriculture is the development of resistance to certain widely used insecticides in a variety of economically relevant insect pests (Pest Manag Sci 2009; 65: 313-322). The application of insecticides to insect pests, that have developed a certain level of resistance to these insecticides, results in crop losses and adds further selective pressure towards additional resistance buildup in these pest populations.

One of the preferred insecticides to control whitefly pests are neonicotinoid insecticides. Neonicotinoid insecticides comprise the following commercialized active ingredients: imidacloprid, thiacloprid, clothianidin, thiamethoxam, acetamiprid, nitenpyram, and dinotefuran. They are also named IRAC Mode of Action class (MoA class) 4A by the Insecticide Resistance Action Committee (IRAC; www.irac-online.org).

Although, farmers would preferably treat whitefly pests with neonicotinoid chemistry, they are faced with a growing number of insect populations that have developed at least some sort of resistance against these active ingredients. Switching fully to other insecticides from different chemistries or different IRAC MoA classes would most likely result in the development of populations of pests being resistant to these insecticides as well. In addition, other chemistries might be inferior in their insecticidal activity.

The standard determination of resistance of pests to specific insecticides currently requires a laboratory environment. Under field or greenhouse conditions, farmers would have to try out an insecticide and then observe if it can control the particular pest of interest. However, the result would be obtained too late, as resistant insects would have already destroyed at least part of the crop. In addition, weak resistance, that already require a resistance management, are not visible under these conditions.

It would therefore be of great practical importance to have an easy to use system, the farmer could directly work in the field, which further achieves a high sensitivity and reliability, and would provide him with a result in minutes, so he can implement proper resistance management strategies based on the rotational use of different mode of action classes not affected by resistance mechanisms.

### DESCRIPTION OF THE INVENTION

### Principle

Surprisingly, levels of cytochrome P450 monooxygenases, in particular CYP6CM1 in combination with an easy to use antibody detection system allow a fast, reliable, and very sensitive detection of resistance of insects, in particular Bemisia tabaci, to neonicotinoid and pymetrozine chemistry.

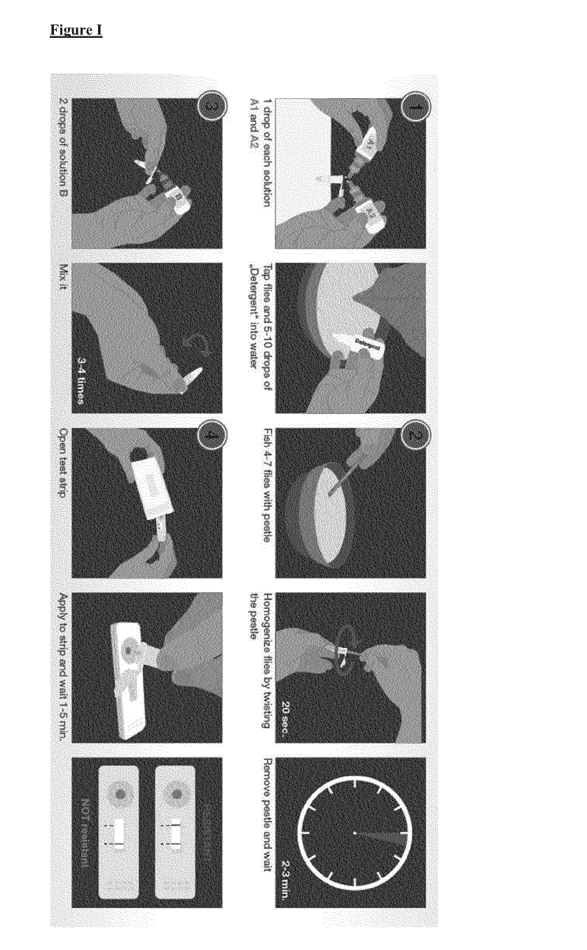

### The Test Kit (See Also FIG. 1)

The basis of the test is the immunological detection, preferably via an ELISA assay, of a P450 monooxygenase, preferably CYP6CM1. The P450 monooxygenase protein is recognized by one or preferably by two different polyclonal antibodies (solutions A1 and A2). Particularly preferred is a combination of an antibody labeled with biotin and an additional antibody labeled with digoxygenin. In the presence of the P450 antigen, the biotinylated antibody will bind additionally to the streptavidin-gold solution (solution B) and form a complex, which now is detected by an anti-digoxygenin antibody spotted as a line on the test strip.

The kit is typically delivered with 3 solutions (A1, A2 and B), single use homogenization vials, single use homogenization pestles and sealed test strips.

The presence of neonicotinoid or pymetrozine resistance can typically be observed by treating 1-20 whiteflies in one whitefly test as described in the test procedure below. An optimum would be around four flies; more than 20 animals would result in a quenched signal, and thus be less preferred.

Whiteflies may be collected in a water bowl, e.g. a plant saucer (ideally of dark color) filled with water and 1-10 drops of detergent (either use commercial dishwashing detergent or 10% Tween20 supplied with the kit). Tapping of flies from the infested plant down into the bowl will collect whiteflies, which float on the surface of the liquid. Flies will stick to the surface of a homogenization pestle when dipped into the bowl.

A general test procedure involves the addition of 1-2 drops of each solution A1 and A2 (each with sufficient amount of the corresponding biotin or digoxygenated labeled antibody, in phosphate buffer and detergent) into the homogenization vial (FIG. 1, Picture 1). Next, the whiteflies are “fished” as described above off the water surface of the bowl. The pestle with 1-20 whiteflies sticking to its tip is inserted into the homogenization vial with liquid (solutions A1 and A2 dropped in before) and twisted carefully in order to homogenize flies (FIG. 1, Picture 2). Homogenization is completed when the solution turns yellowish and turbid. The solution can be used directly for the next step without changing the final result; however, an incubation time of 1-5 min will increase the final signal.

After homogenization, 2 drops of solution B (containing the liquid streptavidin-gold in phosphate buffer and detergent) are added to the vial and the resulting solution is gently mixed by inversion of the vial 3-4 times (FIG. 1, Picture 3). Again, 1-5 min of waiting before proceeding to the next step will increase signal but not change overall result.

For resistance detection, the complete solution is poured from the vial into the sample cavity of the test strip (test strips delivered with the whitefly kit are sealed and have to be removed from the package first) (FIG. 1, Picture 4). A red control line (at the position of “C” marked on the housing) will appear upon flowing of the liquid up the strip, latest after five minutes. Only if this line is present the strip was running properly.

If the control line is present, but no additional test line (at the position of “T” marked on the housing), the tested Bemisia tabaci population is not resistant to neonicotinoids or pymetrozine based on the over-expression of CYP6CM1.

If a clear line at “T” (FIG. 1) is visible, the population is neonicotinoid (and pymetrozine) resistant and an alternative active ingredient of a different mode of action class has to be applied to control those populations.

The detection of the resistance marker can typically be performed with any antibody or antibody derived detection system. The antibody could be either a polyclonal antibody or a monoclonal antibody or a combination of more than one monoclonal antibody. Alternatively, any other molecular high affinity system as e.g. aptamers can be used to work the invention. Preferably, a polyclonal antibody is used. Preferably, combinations of more than one antibody are used for the detection.

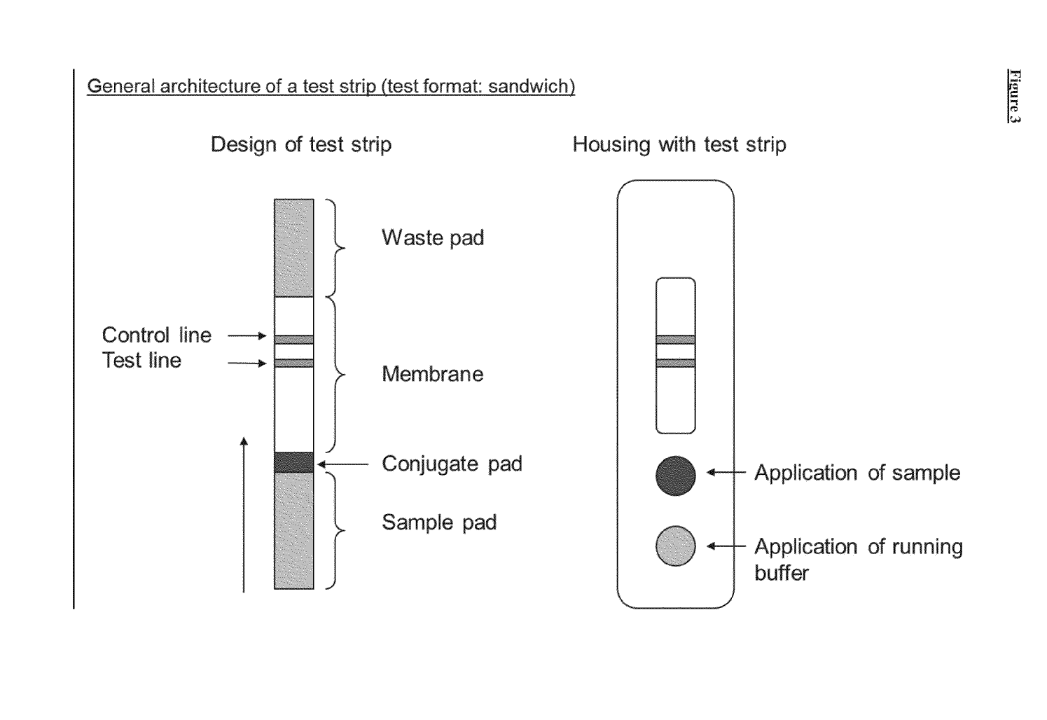

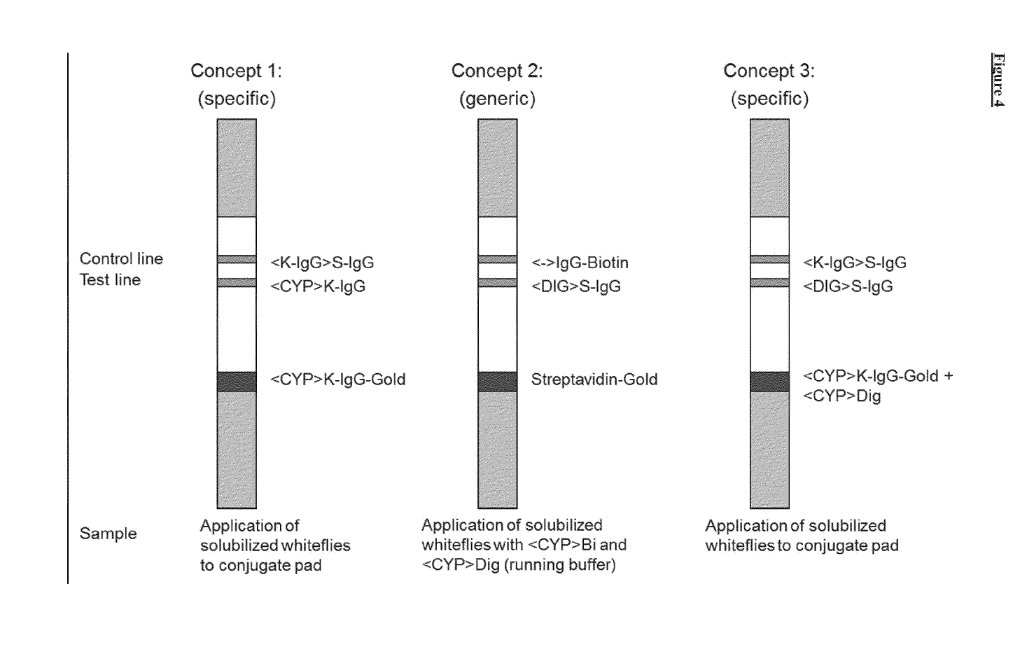

The detection of the resistance marker-antibody complex can typically be performed with any antibody detection system that allows a reasonably easy and quick signal generation as e.g. the digoxygenin detection system or the biotin-streptavidin system. Other suitable detection systems are based on the plasmon resonance principle. Preferably, a combination of a digoxygenin and a biotinylated antibody are preferred. The design of the detection system via a strip (lateral flow) based ELISA is exemplified in FIGS. 3 and 4.

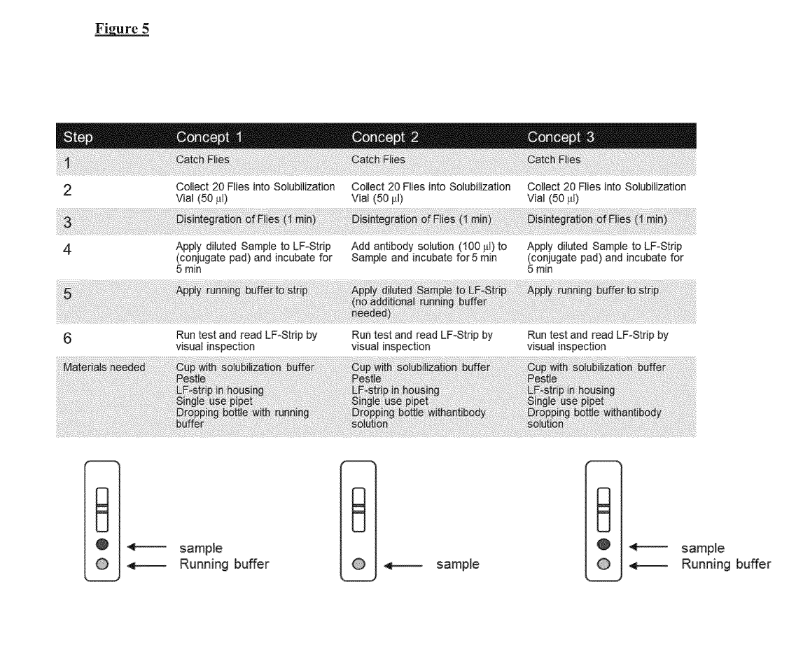

For illustration purposes, three different setups for the ELISA based detection of the resistance marker on strips are displayed in FIG. 5. Preferably the detection of the antibody-resistance marker conjugates is performed via lateral flow analysis.

### The Method for Pest Control

A further embodiment of this invention is a method for controlling insect pests, preferably resistant strains, comprising the following steps:
a. Obtaining a sample of the insect pest,
b. Performing an antibody based detection of a resistance marker selected from the CYP family, preferably via an ELISA assay
c. If the resistance marker is detected, select an insecticide from the list A) consisting of Carbamates, Organophosphates, Cyclodiene organochlorines, Pyrethroids, Sulfoximines, Pyriproxyfen, Diafenthiuron, Benzoylureas, Buprofezin, METI, Tetronic- and Tetramic acids, Butenolides, Diamides, Pyrifluquinazon.
d. If the resistance marker is not present, then select an insecticide either from list A) or the list B) comprised of: imidacloprid, thiacloprid, clothianidin, thiamethoxam, acetamiprid, nitenpyram, and dinotefuran, pymetrozine

Preferred insecticides of List A) for carrying out this invention of the classes Carbamates, Organophosphates, Cyclodiene organochlorines, Pyrethroids, Sulfoximines, Pyriproxyfen, Diafenthiuron, Benzoylureas, Buprofezin, METI, Tetronic- and tetramic acids, Diamides are:

Carbamates: Alanycarb (I1), Aldicarb (I2), Bendiocarb (I3), Benfuracarb (I4), Butocarboxim (I5), Butoxycarboxim (I6), Carbaryl (I7), Carbofuran (I8), Carbosulfan (I9), Ethiofencarb (I10), Fenobucarb (I11), Formetanate (I12), Furathiocarb (I13), Isoprocarb (I14), Methiocarb (I15), Methomyl (I16), Metolcarb (I17), Oxamyl (I18), Pirimicarb (I19), Propoxur (I20), Thiodicarb (I21), Thiofanox (I22), Triazamate (I23), Trimethacarb (I24), XMC (I25), and Xylylcarb (I26);

Organophosphates: Acephate (I27), Azamethiphos (I28), Azinphos-ethyl (I29), Azinphos-methyl (I30), Cadusafos (I31), Chlorethoxyfos (I32), Chlorfenvinphos (I33), Chlormephos (I34), Chlorpyrifos (I35), Chlorpyrifos-methyl (I36), Coumaphos (I37), Cyanophos (I38), Demeton-S-methyl (I39), Diazinon (I40), Dichlorvos/DDVP (I41), Dicrotophos (I42), Dimethoate (I43), Dimethylvinphos (I44), Disulfoton (I45), EPN (I46), Ethion (I47), Ethoprophos (I48), Famphur (I49), Fenamiphos (I50), Fenitrothion (I51), Fenthion (I52), Fosthiazate (I53), Heptenophos (I54), Imicyafos (I55), Isofenphos (I56), Isopropyl O-(methoxyaminothio-phosphoryl) salicylate (I57), Isoxathion (I58), Malathion (I59), Mecarbam (I60), Methamidophos (I61), Methidathion (I62), Mevinphos (I63), Monocrotophos (I64), Naled (I65), Omethoate (I66), Oxydemeton-methyl (I67), Parathion (I68), Parathion-methyl (I69), Phenthoate (I70), Phorate (I71), Phosalone (I72), Phosmet (I73), Phosphamidon (I74), Phoxim (I75), Pirimiphos-methyl (I76), Profenofos (I77), Propetamphos (I78), Prothiofos (I79), Pyraclofos (I80), Pyridaphenthion (I81), Quinalphos (I82), Sulfotep (I83), Tebupirimfos (I84), Temephos (I85), Terbufos (I86), Tetrachlorvinphos (I87), Thiometon (I88), Triazophos (I89), Trichlorfon (I90), and Vamidothion (I91);

Cyclodiene organochlorines: Chlordane (I92) and Endosulfan (I93);

Pyrethroids: Acrinathrin (I96), Allethrin (I97), d-cis-trans Allethrin (I98), d-trans Allethrin (I99), Bifenthrin (I100), Bioallethrin (I101), Bioallethrin S-cyclopentenyl isomer (I102), Bioresmethrin (I103), Cycloprothrin (I104), Cyfluthrin (I105), beta-Cyfluthrin (I106), Cyhalothrin (I107), lambda-Cyhalothrin (I108), gamma-Cyhalothrin (I109), Cypermethrin (I110), alpha-Cypermethrin (I111), beta-Cypermethrin (I112), theta-Cypermethrin (I113), zeta-Cypermethrin (I114), Cyphenothrin [(1R)-trans isomers] (I115), Deltamethrin (I116), Empenthrin [(EZ)-(1R) isomers) (I117), Esfenvalerate (I118), Etofenprox (I119), Fenpropathrin (I120), Fenvalerate (I121), Flucythrinate (I122), Flumethrin (I123), tau-Fluvalinate (I124), Halfenprox (I125), Imiprothrin (I126), Kadethrin (I127), Permethrin (I128), Phenothrin [(1R)-trans isomer) (I129), Prallethrin (I130), Pyrethrine (pyrethrum) (I131), Resmethrin (I132), Silafluofen (I133), Tefluthrin (I134), Tetramethrin (I135), Tetramethrin [(1R) isomers)] (I136), Tralomethrin (I137), and Transfluthrin (I138); or DDT (I139); or Methoxychlor (I140);

Benzoylureas: Bistrifluron (I191), Chlorfluazuron (I192), Diflubenzuron (I193), Flucycloxuron (I194),

Flufenoxuron (I195), Hexaflumuron (I196), Lufenuron (I197), Novaluron (I198), Noviflumuron (I199), Teflubenzuron (I200), and Triflumuron (I201);

METI: Fenazaquin (I212), Fenpyroximate (I213), Pyrimidifen (I214), Pyridaben (I215), Tebufenpyrad (I216), and Tolfenpyrad (I217); or Rotenone (Derris) (I218);

Tetronic and tetramic acids: e.g. Spirodiclofen (I221), Spiromesifen (I222), and Spirotetramat (I223);

(25) Mitochondrial complex II electron transport inhibitors, for example beta-ketonitrile derivatives, e.g. Cyenopyrafen (I229) and Cyflumetofen (I230);

Diamides: Chlorantraniliprole (I231), Cyantraniliprole (I232), and Flubendiamide (I233);

Butenolides: 4-{[(6-bromopyridin-3-yl)methyl](2-fluoroethyl)amino}furan-2(5H)-one (I258) (known from WO2007/115644), 4-{[(6-fluoropyridin-3-yl)methyl](2,2-difluoroethyl)amino}furan-2(5H)-one (I259) (known from WO2007/115644), 4-{[(2-chloro-1,3-thiazol-5-yl)methyl](2-fluoroethyl)amino}furan-2(5H)-one (I260) (known from WO2007/115644), 4-{[(6-chlorpyridin-3-yl)methyl](2-fluoroethyl)amino}furan-2(5H)-one (I261) (known from WO2007/115644), Flupyradifurone (I262), 4-{[(6-chlor-5-fluoropyridin-3-yl)methyl](methyl)amino}furan-2(5H)-one (I263) (known from WO2007/115643), 4-{[(5,6-dichloropyridin-3-yl)methyl](2-fluoroethyl)amino}furan-2(5H)-one (I264) (known from WO2007/115646), 4-{[(6-chloro-5-fluoropyridin-3-yl)methyl](cyclopropyl)amino}furan-2(5H)-one (I265) (known from WO2007/115643), 4-{[(6-chloropyridin-3-yl)methyl](cyclopropyl)amino}furan-2(5H)-one (I266) (known from EP-A-0 539 588), 4-{[(6-chlorpyridin-3-yl)methyl](methyl)amino}furan-2(5H)-one (I267) (known from EP-A-0 539 588),

Sulfoxymines: {[1-(6-chloropyridin-3-yl)ethyl](methyl)oxido-λ4-sulfanylidene}cyanamide (I268) (known from WO2007/149134) and its diastereomers {[(1R)-1-(6-chloropyridin-3-yl)ethyl](methyl)oxido-λ4-sulfanylidene}cyanamide (A) (I269), and {[(1S)-1-(6-chloropyridin-3-yl)ethyl](methyl)oxido-λ4-sulfanylidene}cyanamide (B) (I270) (also known from WO2007/149134) as well as diastereomers [(R)-methyl(oxido){(1R)-1-[6-(trifluoromethyl)pyridin-3-yl]ethyl}-λ4-sulfanylidene]cyanamide (A1) (I271), and [(S)-methyl(oxido){(1S)-1-[6-(trifluoromethyl)pyridin-3-yl]ethyl}-λ4-sulfanylidene]cyanamide [also known as sulfoxaflor] (A2) (I272), referred to as group of diastereomers A (known from WO2010/074747, WO2010/074751), [(R)-methyl(oxido){(1S)-1-[6-(trifluoromethyl)pyridin-3-yl]ethyl}-λ4-sulfanylidene]cyanamide (B1) (I273), and [(S)-methyl(oxido){(1R)-1-[6-(trifluoromethyl)pyridin-3-yl]ethyl}-λ4-sulfanylidene]cyanamide

More preferably, list A) consists of spirotetramat, spiromesifen and flupyradifurone. Very preferably, list A) consists of spirotetramat and spiromesifen.

Preferably, list B) consists of imidacloprid or pymetrozine.

A preferred resistance marker is CYP6CM1.

A preferred insect pest is of the genus Bemisia. A very preferred insect pest is the cotton whitefly (Bemisia tabaci).

The active ingredients specified herein by their “common name” are known and described, for example, in the Pesticide Manual (“The Pesticide Manual”, 14th Ed., British Crop Protection Council 2006) or can be searched in the internet (e.g. http://www.alanwood.net/pesticides).

### Preparation and Composition of Reagents:

### Immunization of Rabbits:

4 rabbits were immunized with His6-CYP6CM1 in PBS (500 μg per injection).

The immunization of a SPF rabbit (SPF=Specific Pathogen Free; strain: New Zealand White) includes:
Immunization standard protocol (4 injections at day 0, 28, 42, 56), 2 control (after 40 and 54 days)
Serum of pre-bleed (before first injection)
Immunization is carried out by subcutaneous injections in combination with adjuvants (first injection CFA, second and further injections IFA)
Delivery of serum (1. and 2. bleed 3-5 mL each, at day 70 bleed of about 20 ml each, then every month bleed of about 20 ml of each rabbit)

### Purification of Antibodies:

The antibody was purified from serum by positive immunosorption. To prepare the column matrix, the antigen MBP-CYP6CM1 was coupled to NHS-activated Sepharose 4FF (GE Healthcare) following the instructions of the manufacturer. The purification was performed by binding the portion of antibody specific to CYP6CM1 by pumping the serum through a column filled with MBP-CYP6CM1-Sepharose. After two washing steps with (1) 0.5 M NaCl, 0.05% Tween 20 and (2) 30 mM NaCl the specific antibody was eluted under acidic conditions with a buffer containing 100 mM glycine, 0.5 M NaCl, pH 2.7. The antibody was dialyzed against PBS and concentrated by ultrafiltration.

### Modification of Antibodies:

The antibodies were labelled by coupling NHS-activated labelling reagents that reacts efficiently with primary amino groups (—NH2) to form stable amide bonds. Antibodies have several primary amines in the side chain of lysine (K) residues and the N-terminus of each polypeptide that are available as targets for labeling with NHS-activated reagents. Out of these available amino groups, some residues are randomly coupled during reaction. The degree of labelling is dependent on the molar ratio used during conjugation reaction.

The biotinylation was performed in 100 mM phosphate buffer pH 8.5 with 2 to 8-fold molar excess of biotinylation reagent (succinimidyl-6-(biotinamido)hexanoate) at 25° C. for two hours. The reaction was stopped by adding 10 mM lysine. The conjugate was purified from excess biotinylation reagent and by-products by dialysis against PBS. The degree of labelling was determined by a HABA assay.

The conjugation with Digoxigenin was performed in 100 mM phosphate buffer pH 8.5 with 8-fold molar excess of labeling reagent (Digoxigenin-3-O-methylcarbonyl-e-aminocaproic acid-N-hydroxysuccinimide ester) at 25° C. for two hours. The reaction was stopped by adding 10 mM lysine. The conjugate was purified from excess labelling reagent and by-products by dialysis against PBS.

### Homogenisation Buffer:

| Ingredient | Range | | Particularly preferred | |
|---|---|---|---|---|
| Sodium Phosphate or any other buffer | 0.1-10 mM, pH 6.0 to 8.0 | | 1 mM, pH 6.7 | |
| system for a pH range of 6.0 to 8.0 | | | | |
| Detergent 1: e.g. Triton X-100 | 0.02-0.5% | per weight | 0.1% | per weight |
| Detergent 2: Dodecyl-β-D-maltosid | 0.5-3% | per weight | DDM 1% | per weight |
| (DDM), alternatively: n-Octyl- β-D- | | | | |
| glucopyranosid (OG), 3-[(3- | | | | |
| Cholamidopropyl)dimethylammonio]- | | | | |
| 1-propansulfonat (CHAPS), | | | | |
| sodiumcholate, Polyethylenglykol | | | | |
| 40000 (PEG40) | | | | |
| Optional: Saccharose or any other | 0-250 | mM | 150 | mM |
| suitable reagent to control the | | | | |
| osmolarity | | | | |

### Running Buffer:

| Ingredient | Range | | Particularly preferred | |
|---|---|---|---|---|
| Sodium Phosphate or any other | 0.1-10 mM, pH 7.5 to 9.0 | | 1 mM, pH 8.0 | |
| buffer system for a pH range of | | | | |
| pH 7.5 to 9.0 | | | | |
| Detergent 1: e.g. Triton X-100 | 0.01-0.2% | per weight | 0.05% | per weight |
| Bovine Serum Albumine or any | 0.05-1% | per weight | 0.1% | per weight |
| other appropriate agent to | | | | |
| prevent unspecific binding | | | | |
| Optional: Saccharose or any | 0-250 | mM | 150 | mM |
| other suitable reagent to control | | | | |
| the osmolarity | | | | |

### Antibody:

50 ng-500 ng, preferably 150 ng each (Antibody biotin and antibody digoxygenin conjugate)

### Streptavidin-Gold (40 nm):

100 mOD 50-300 mOD

### Teststrip for Lateral Flow Application (See Also FIGS. 3 and 4):

Membrane card: e.g. HF120 Plus (Millipore)

Waste Pad: C083 (Millipore) or any other similar cellulosepads.

Sample Pad: C083 (Millipore), treated with a solution comprising:

a) 100-400 mM Borate containing buffer, pH 8.0-10.0, preferably 250 mM sodium borate, pH 9.0;

b) 0.1-3% per weight BSA, preferably 1% per weight BSA;

c) 0.01-0.2% per weight Triton X-100, preferably 0.05% Triton X-100

### Example

By utilization of the detection kit, a set of Bemisia tabaci strains was tested and susceptibility/resistance detected in the laboratory:

1) ESP-07M: resistant, Q-type, Spain

2) ESP-00: resistant, less resistant than 1), Q-type, Spain

3) SuS: susceptible, neither B nor Q-type, Sudan

4) B-Ref: susceptible, B-type, Israel

5) Crete: resistant, less resistant than 1) and 2), Q-type, Greece

6) EAE: resistant, B-type, Brazil

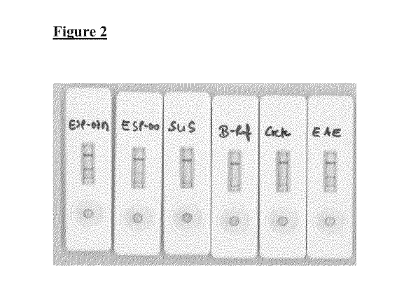

In this test strains 1, 2, 5 and 6 were positively tested for the resistance marker by using the kit (FIG. 2). In all cases, the detection of the resistance marker correlated with the resistance against imidacloprid.

1. Method for controlling one or more insect pests comprising:
a. Obtaining a sample of the insect pest,
b. Performing an antibody based detection of a resistance marker selected from the CYP family,
c. If the resistance marker is detected, select an insecticide from the list A) comprising Carbamates, Organophosphates, Cyclodiene organochlorines, Pyrethroids, Sulfoximines, Pyriproxyfen, Diafenthiuron, Benzoylureas, Buprofezin, METI, Tetronic- and Tetramic acids, Butenolides, Diamides, Pyrifluquinazon.
d. If the resistance marker is not present, then select an insecticide either from list A) or from the list B) comprising imidacloprid, thiacloprid, clothianidin, thiamethoxam, acetamiprid, nitenpyram, dinotefuran, pymetrozine.

2. Method according to claim 1, wherein the insect pest is of the genus Bemisia, optionally of the species Bemisia tabaci.

3. Method according to claim 1, wherein the resistance marker is CYP 6CM1.

4. Method to claim 1, wherein list A) comprises spirotetramat, spiromesifen and flupyradifurone.

5. Method of claim 1, wherein list A) comprises spirotetramat and spiromesifen.

6. Kit for the determination of resistance of an insect pest species towards a specific insecticide comprising the following components:
a. A solubilization solution,
b. An antibody specific for a cytochrome P450 monooxygenase
c. A detection system for the antibody of b.

7. CYP 6CM1 detection that is capable of being used for resistance management in whitefly.
